(19) United States
(12) Patent Application Publication
(10) Pub. No.: US 2010/0111889 A1
(43) Pub. Date: May 6, 2010

(54) Malodor Control System

(76) Inventors: Randall Glenn Marsh, Fairfield Township, OH (US); Ricky Ah-Man Woo, Liberty Township, OH (US); John David Bauer, Finneytown, OH (US); Carianne Michelle Duncan, Cincinnati, OH (US); Jackson Lynn Cummins, Independence, KY (US); Judith Ann Hollingshead, Batavia, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/611,310

(22) Filed: Nov. 3, 2009

Related U.S. Application Data
(60) Provisional application No. 61/111,105, filed on Nov. 4, 2008.

(51) Int. Cl. A61L 9/01

(52) U.S. Cl. 424/76.1

(57) ABSTRACT

A malodor control system suitable for use in disposable articles, such as disposable cleaning wipes, baby wipes, or skin care wipes, is disclosed. The system may comprise an aldehyde, an ester, an ionone, and optionally a macrocyclic musk. The system may optionally include a perfume.

### CROSS REFERENCE TO RELATED APPLICATION

This application claims the benefit of U.S. Provisional Application No. 61/111,105, filed Nov. 4, 2008, which is herein incorporated by reference in its entirety.

### FIELD OF THE INVENTION

The present disclosure relates to a malodor control system which is suitable for a variety of applications, including use in disposable wipes.

### BACKGROUND OF THE INVENTION

Products for reducing or masking malodors are well known in the art and widely described in the patent literature. These products may be designed to work specifically in air or on fabrics or other surfaces. See, for example, U.S. Pat. Nos. 5,942,217; 5,955,093; and 6,033,679.

Disposable absorbent articles have proven a difficult challenge for odor reduction technologies. Bowel movement and urine odors are typically unpleasant, and can arise from a variety of underlying compounds, depending upon the wearer's diet, environment, genetics, health, and other factors. Similarly, menses, perspiration, wound drainage, and other bodily exudates may have an undesirable odor on excretion or may develop malodor when contained for some period of time by a disposable absorbent article.

The difficulty in overcoming such odors has spawned a diverse assortment of products to eliminate, neutralize, reduce, mask, or contain the malodors associated with soiled disposable absorbent articles. Scented products are widely available, including scented garbage bags, sprays, disinfectant sprays, air freshener sprays, and powders, for use in a diaper or in a pail used to hold soiled diapers.

Even effective air-dispersed odor neutralization technologies have limited effectiveness in the particular context of soiled disposable absorbent articles, because air-dispersed technologies do not prevent a caregiver's exposure to malodor while changing, for example, a soiled diaper, soiled wound dressing, soiled perspiration-absorbent pad, or soiled feminine hygiene product. Further, a caregiver may be hesitant to spray chemicals onto skin contaminated with a malodorous exudate, especially, but not exclusively, when dealing with infants and/or with the genital and perianal anatomy.

There remains a need for an effective malodor control system for confined odor sources, such as those associated with disposable absorbent articles, which is not reliant on overpowering a malodor with an overwhelming perfume, and which does not require the purchase and deployment of additional goods, such as sprays, powders, special disposal equipment or supplies, and the like.

### BRIEF DESCRIPTION OF THE DRAWINGS

FIG. 1 illustrates a Schiff-base reaction between malodorous aminic compounds and aldehydes.

FIG. 2 illustrates an aminolysis reaction between malodorous aminic compounds and selected ester compounds.

FIG. 3 is a graph showing the effects of various optional components of a malodor control system on malodor on a cleaned surface.

FIG. 4 is a graph showing the effects of various optional components of a malodor control system on malodor on a wipe used to clean a malodorous surface.

FIG. 5 is a graph showing the effects of various malodor technologies on malodor on a cleaned surface.

FIG. 6 is a graph showing the effects of various malodor technologies on malodor on a wipe used to clean a malodorous surface.

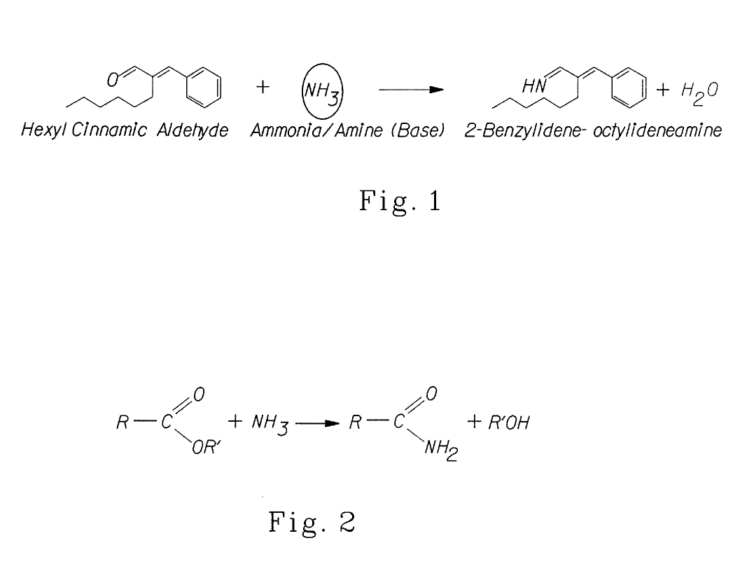

### DETAILED DESCRIPTION OF THE INVENTION

As follows, a malodor control system is described particularly in regard to a wipe, such as a baby wipe or a skin cleansing wipe. A malodor control system is also applicable to products with different purposes, such as wipes for pets, wipes for cleaning fabrics or upholstery, wipes for surface cleaning, etc., which in most cases would require only routine adjustments to the formulas described.

As used herein, the following terms have the described meanings, which may be consistent or inconsistent with common usage or usage in the art:

“Perfume” refers to a compound utilized for its appealing odor. Compounds may have a pleasing odor without being used as a “perfume” in the context of this disclosure.

“Odor-neutralization” refers to the ability of a compound or product to eliminate malodorous compounds. Odor-neutralization may be partial, affecting only some of the malodorous compounds in a given context, or affecting only part of a malodorous compound. A malodorous compound may be “eliminated” by chemical reaction resulting in a new chemical entity, by sequestration, by chelation, by association, or by any other interaction rendering the malodorous compound less malodorous or non-malodorous. Odor-neutralization may be distinguished from odor-masking or odor-reducing by a change in the malodorous compound(s), as opposed to a change in the ability to perceive the malodor without any corresponding change in the condition of the malodorous compound(s).

“Odor-masking” refers to the ability of a compound to mask or hide a malodorous compound. Odor-masking may be differentiated from odor-overwhelming, wherein a compound with a non-offensive or pleasant smell is dosed such that it dominates the partial pressure of other compounds in a given airspace and therefore limits the ability to sense a malodorous compound or any other compound. Odor-masking may involve the selection of compounds which coordinate with an anticipated malodor to change the perception of the overall scent provided by the combination of odorous compounds.

“Odor-reducing” or “anosmia-inducing” refers to the ability of a compound to dull the human sense of smell. Anosmia may be induced with regard to only specific smells or types of smells, or more generally, with regard to a broad range of smells or even to all smells.

“Scent” or “scented” refer to a compound having a detectable odor at relevant concentrations that would generally be non-offensive or pleasant to most people. “Malodor” or “malodorous” refer to compounds generally offensive or unpleasant to most people, such as the complex odors associated with bowel movements.

### Malodor Control System

Malodors encountered while using a baby wipe can arise from a number of sources. The wipe may be used to remove urine and fecal residues from the infant's body, and those residues may be malodorous inherently or may become malodorous after changes, as, for example, from chemical reactions or bacterial growth following excretion, or both. A disposable absorbent article, such as a diaper, and even the wipe itself may also have undesirable odors arising from interactions or reactions between the components or off-gassing of components of the diaper or wipe. These odors cannot effectively be addressed by air fresheners or even air-dispersed odor neutralizers because they are not maximally airborne until a diaper change is underway. Using an air-dispersed product during a diaper change is undesirable for several reasons. For example, using a separate product complicates the procedure of diaper changing, which can be sufficiently challenging with a mobile, fussy, or heavily soiled infant or toddler that the use of an air freshener is not realistically possible. Further, many caregivers may resist deploying an inhalable mist of chemicals over a young child during each diaper change.

In some embodiments of the present disclosure, a malodor control system is provided in or associated with a baby wipe, rendering the wipe capable of reducing malodors. In some embodiments, the malodor control system is compatible with perfumes and other lotion components, such that the malodor control system does not interfere with other properties of the wipe, including, if desired, a pleasant smell associated with the wipe itself.

In one embodiment, a malodor control system is provided that can both neutralize and mask malodors associated with a diaper change without interfering with other typical components in a wipe liquid phase formula, including one or more adjunct perfumes. Odor neutralization may be provided using ingredients that interact with and, through chemical reactions or sequestration, isolate or chemically or physically alter the malodor-producing compounds. For example, urine malodor may arise from di- and tri-sulfides, acids, amines and amides, heterocyclic nitrogenous compounds, aldehydes, ketones, substituted aromatic compounds, alcohols and esters; and fecal malodor may arise from short chain fatty acids, aldehydes, alcohols, ketones, esters, sulfur compounds, and nitrogen compounds. It is therefore possible to select compounds which neutralize the malodor(s) associated with urine and fecal matter by providing compounds which interact with the malodorous compounds to neutralize the malodor. For example, certain aldehyde and ester compounds may interact with malodor-producing compounds, resulting in low-odor or organoleptically inert reaction products.

Without wishing to be bound by theory, it is believed that three predominant reactions are involved in odor neutralization. Some compounds, such as cinnamic aldehyde, decanal, and ammonia participate in Schiff-base reactions resulting in organoleptically inert compounds. Other compounds, such as linalyl acetic acid, benzyl benzoic acid, methyl abietic acid, and ammonia, may undergo acid-base neutralization as a consequence of ester hydrolysis, again resulting in low-odor or organoleptically inert compounds. Still other compounds, such as ester compounds like triethyl citrate and hexyl salicylate, may participate in aminolysis reactions, yet again resulting in low-odor or organoleptically inert compounds. In each case, the end result is the neutralization of the malodor-causing compound(s).

Exemplary aldehydes which may be used in a malodor control system include, but are not limited to, Adoxal (2,6,10-trimethyl-9-undecenal), Bourgeonal (4-t-butylbenzenepropionaldehyde), cinnamic aldehyde, cinnamaldehyde (phenyl propenal, 3-phenyl-2-propenal), citral, Geranial, Neral (dimethyloctadienal, 3,7-dimethyl-2,6-octadien-1-al), cyclal C (2,4-dimethyl-3-cyclohexen-1-carbaldehyde), florhydral (3-(3-Isopropyl-phenyl)-butyraldehyde), citronellal (3,7-dimethyl 6-octenal), cymal cyclamen aldehyde, cyclosal, lime aldehyde (alpha-methyl-p-isopropyl phenyl propyl aldehyde), methyl nonyl acetaldehyde, aldehyde C12 mna (2-methyl-1-undecanal), hydroxycitronellal, citronellal hydrate (7-hydroxy-3,7-dimethyl octan-1-al), helional (alpha-methyl-3,4-(methylenedioxy)-hydrocinnamaldehyde), hydrocinnamaldehyde (3-phenylpropanal, 3-phenylpropionaldehyde), intreleven aldehyde (undec-10-en-1-al), ligustral, trivertal (2,4-dimethyl-3-cyclohexene-1-carboxaldehyde), Jasmorange, satinaldehyde (2-methyl-3-tolylpropionaldehyde, 4-dimethylbenzenepropanal), lyral (4-(4-hydroxy-4-methyl pentyl)-3-cyclohexene-1-carboxaldehyde), melonal (2,6-dimethyl-5-heptenal), methoxy melonal (6-methoxy-2,6-dimethylheptanal), methoxycinnamaldehyde (trans-4-methoxycinnamaldehyde), myrac aldehyde (isohexenyl cyclohexenyl-carboxaldehyde), trifernal (3-methyl-4-phenyl propanal, 3-phenyl butanal), lilial (P.T. Bucinal, Lilestralis 33, p-t-butyl-a-methylhydrocinnamic aldehyde, 2-methyl-3-(4-t-butylphenyl)propanal, lysmeral), benzenepropanal (4-tert-butyl-alpha-methyl-hydrocinnamaldehyde), dupical, tricyclodecylidenebutanal (4-Tricyclo5210-2,6decylidene-8butanal), melafleur (1,2,3,4,5,6,7,8-octahydro-8,8-dimethyl-2-naphthaldehyde), methyl octyl acetaldehyde, aldehyde C-11 MOA (2-methyl decan-1-al), oncidal (2,6,10-trimethyl-5,9-undecadien-1-al), cintronellyl oxyacetaldehyde, Muguet Aldehyde 50 (3,7-dimethyl-6-octenyl) oxyacetaldehyde), phenylacetaldehyde, mefranal (3-methyl-5-phenyl pentanal), triplal, vertocitral dimethyltetrahydrobenzenealdehyde (2,4-dimethyl-3-cyclohexene-1-carboxaldehyde), 2-phenylpropionaldehyde, hydrotropaldehyde, canthoxal, p-anisic alcohol, ansisylpropanal 4-methoxy-alpha-methyl benzenepropanal (2-anisylidene propanal), cylcemone A (1,2,3,4,5,6,7,8-octahydro-8,8-dimethyl-2-naphthaldehyde), and precylcemone B (1-cyclohexene-1-carboxaldehyde).

Still other exemplary aldehydes include, but are not limited to, acetaldehyde (ethanal), pentanal, valeraldehyde, amylaldehyde, scentenal (octahydro-5-methoxy-4,7-Methano-1H-indene-2-carboxaldehyde), propionaldehyde (propanal), cyclocitral, beta-cyclocitral, (2,6,6-trimethyl-1-cyclohexene-1-acetaldehyde), iso cyclocitral (2,4,6-trimethyl-3-cyclohexene-=1-carboxaldehyde), isobutyraldehyde, butyraldehyde, isovaleraldehyde (3-methyl butyraldehyde), methylbutyraldehyde (2-methyl butyraldehyde, 2-methyl butanal), dihydrocitronellal (3,7-dimethyl octan-1-al), 2-ethylbutyraldehyde, 3-Methyl-2-butenal, 2-methylpentanal, 2-methyl valeraldehyde, hexenal (2-hexenal, trans-2-hexenal), heptanal, octanal, nonanal, decanal, lauric aldehyde, tridecanal, 2-dodecanal, methylthiobutanal, glutaraldehyde, pentanedial, glutaric aldehyde, heptenal, cis or trans-heptenal, undecenal (2-, 10-), 2,4-octadienal, nonenal (2-, 6-), decenal (2-, 4-), 2,4-hexadienal, 2,4-decadienal, 2,6-nonadienal, octenal, 2,6-dimethyl 5-heptenal, 2-isopropyl-5-methyl-2-hexenal, beta methyl benzenepropanal, 2,6,6-Trimethyl-1-cyclohexene-1-acetaldehyde, phenyl butenal (2-phenyl 2-butenal), 2.Methyl-3(p-isopropylphenyl)-propionaldehyde, 3-(p-isopropylphenyl)-propionaldehyde, p-tolylacetaldehyde (4-methylphenylacetaldehyde), anisic aldehyde, anisaldehyde (p-methoxybenzene aldehyde), benzaldehyde, vernaldehyde (1-Methyl-4-(4-methylpentyl)-3-cyclohexenecarbaldehyde), heliotropin (piperonal) 3,4-methylene dioxy benzaldehyde, alpha-amylcinnamic aldehyde, 2-pentyl-3-phenylpropenoic aldehyde, vanillin (4-methoxy 3-hydroxy benzaldehyde), ethyl vanillin (3-ethoxy 4-hydroxybenzaldehyde), hexyl cinnamic aldehyde, Jasmonal H (alpha-n-hexyl-cinnamaldehyde), floralozone, (para-ethyl-alpha,alpha-dimethyl hydrocinnamaldehyde), acalea (p-methyl-alpha-pentylcinnamaldehyde), methylcinnamaldehyde, alpha-methylcinnamaldehyde (2-methyl 3-pheny propenal), alpha-hexylcinnamaldehyde (2-hexyl 3-phenyl propenal), salicylaldehyde (2-hydroxy benzaldehyde), 4-ethyl benzaldehyde, cuminaldehyde (4-isopropyl benzaldehyde), ethoxybenzaldehyde, 2,4-dimethylbenzaldehyde, veratraldehyde (3,4-dimethoxybenzaldehyde), syringaldehyde (3,5-dimethoxy 4-hydroxybenzaldehyde), catechaldehyde (3,4-dihydroxybenzaldehyde), safranal (2,6,6-trimethyl-1,3-diene methanal), myrtenal (pin-2-ene-1-carbaldehyde), perillaldehyde L-4(1-methylethenyl)-1-cyclohexene-1-carboxaldehyde), 2,4-Dimethyl-3-cyclohexene carboxaldehyde, 2-Methyl-2-pentenal, 2-methylpentenal, pyruvaldehyde, formyl tricyclodecan, mandarin aldehyde, cyclemax, pino acetaldehyde, corps iris, maceal, and corps 4322.

Exemplary esters which may be used in a malodor control system include, but are not limited to, Menthyl Acetate, Ethyl Propionate, Butyl Acetate, Pyruvic Acetate, Methyl Malonate, Prenyl Acetate, Hexyl Formate, Ethyl-3-Hydroxy Butyrate (Ethoxiff), Manzanate, Butyl iso-Butyrate, Ethyl Aceto Acetate, iso-Amyl Propionate, 1-Ethoxy-2-Methyl-Propane, Hexyl Acetate, Lime Oxide, iso-Butyl Angelate, DiEthyl Malonate, Allyl Caproate, Benzyl Formate, Inonyl Formate (PPF), Hexyl Propionate, Octenyl Acetate, 3-Octanol, acetate, Hexyl iso-Butyrate, iso-Amyl Angelate, Benzyl Acetate, iso-Nonyl Acetate, Ethyl Benzoate, Phenyl Ethyl Formate, Methyl Phenyl Acetate, Ethyl Octanoate, Methyl Salicylate, Tetra Hydro Linalyl Acetate, trans-Hexenyl iso-Valerate, Allyl Amyl Glycolate, Ethyl Phenyl Acetate, Linalyl Acetate, Iso-Nonyl Propionate, Phenyl Ethyl Acetate, Tetrahydro Lavandulyl Acetate, Benzyl Propionate, Myrcenyl Acetate, Bergamyl Acetate, iso-Pulegyl Acetate Major, Methyl Phenyl Carbinyl Propionate, Ethyl Nonanoate, Ethyl Salicylate, Lavandulyl Acetate, beta-Terpinyl Acetate, Ocimenyl Acetate (cis or trans), iso-Pulegyl Acetate Isomer, Bornyl Acetate, iso-Bornyl Acetate, Phenyl Ethyl Propionate, Nonyl Acetate, and Methyl Geraniate.

Other exemplary esters include, but are not limited to, DiHydro Terpinyl Acetate, Dimethyl Benzyl Carbinyl Acetate, Myrtenyl Acetate, iso-DiHydro Lavandulyl Acetate, Ethyl Linalyl Acetate, 1,2,3-Propanetriol Diacetate, Linalyl Propionate, 4-tert butyl cyclohexyl aceate, iso-Butyl Benzoate, Citronellyl Acetate, alpha-Terpinyl propionate, Ethyl Linalyl Acetate, Neryl Acetate, Carvyl Acetate, Terpinyl Acetate, Bergamyl Acetate, Allyl Nonanoate, Octyl Acetate, Linalyl iso-Butyrate, Geranyl Acetate, Butyl Benzoate, Hexyl Caproate, Ethyl Geranate, Methyl Cinnamate, iso-Butyl Phenyl Acetate, Sulfuryl Acetate, iso-Bornyl Propionate, Phenyl Ethyl iso-Butyrate, Methyl Eugenol, Ethyl-o-Methoxy Benzoate, Koumalactone, Longicyclene, beta-Terpinyl-iso-Butyrate, Linalyl Butyrate, Anisyl Acetate, Allyl Cyclo Hexane Propionate, Phenyl Ethyl Butyrate, Hexyl Crotanate, Flor Acetate, iso-Amyl Caprylate, iso-Butyl Salicylate, Nopyl Acetate, Butyl Salicylate, Citronellyl Propionate, iso-Amyl Benzoate, iso-Bornyl iso-Butyrate (Abierate CN), Phenyl Ethyl DiMethyl, Carbinyl Acetate, Acetate PA (Allyl Phenoxy Acetate), Cinnamyl Acetate, DiHydro Cyclacet, Ethyl-2,4-Decadienoate, Geranyl Propionate, Veticol Acetate, Ethyl Cinnamate, Phenoxy Ethyl Propionate, gamma-Terpinyl-iso-Butyrate, Citronellyl iso-Butyrate, Ethyl Undecylenate, Neryl iso-Butyrate, Amyl Benzoate, and Decyl Propionate.

Still other exemplary esters include, but are not limited to Prenyl Benzoate (Proflora IFF), Dimethyl Benzyl Carbinyl Butyrate, Phenoxy Ethyl iso-Butyrate, Ethyl Phenyl Glycidate, Eugenyl Acetate, Frutene, Cinnamyl Propionate, Amyl Salicylate, iso-Amyl Phenyl Acetate, Geranyl Butyrate, Ethyl Dodecanoate, Hexyl Benzoate, Cinnamyl iso-Butyrate, Amyl Salicylate, Phenyl Ethyl Tiglate, Linalyl Caproate, Prenyl Salicylate, DiEthyl Phthalate, iso-Eugenyl Acetate, Citronellyl Valerate, iso-Amyl Caproate, TriEthyl Citrate, Methyl Dihydro Jasmonate (cis), Methyl Jasmonate, Citronellyl Tiglate, Iso-Butavan/iso-Butyl Lignate, Arbrensa (IFF), DiBenzyl Ether (Arbrensa IFF), Dupical, Lyral, Phenyl Benzoate, Methyl Dihydro Jasmonate (trans), Geranyl Tiglate, Hexyl Salicylate, Citronellyl Caproate, Methyl Myristate, Myraldyl Acetate, Caryophyllene Acetate, iso-Amyl Undecylenate, Geranyl Caproate, Cyclohexyl Salicylate, Benzyl Benzoate, Ethyl Myristate, Linalyl Caprylate, Cedryl Acetate, Amboryl Acetate, Linalyl Benzoate, Isopropyl Myristate, Benzyl Phenyl Acetate, Guaiacwood Acetate, Vetivert Major, iso-Amyl Laurate, Phenyl Ethyl Benzoate, Benzyl Salicylate, Methyl Palmitate, Citronellyl Caprylate, Neryl Caprylate, Phenyl Ethyl Phenyl Acetate, Citronellyl Benzoate, Geranyl Caprylate, Geranyl Benzoate, Phenyl Ethyl Salicylate, Citronellyl Phenyl Acetate, Ethyl Palmitate, Citronellyl Pelargonate, Isopropyl Palmitate, Geranyl Phenyl Acetate, Geranyl Pelargonate, Ethylene Brassylate (Musk T), Anisyl Phenyl Acetate (Timberiff), C21 Hydrocarbon, Jasmine Absolute, Benzyl Cinnamate (Peru Balsam), Benzyl iso-Eugenol, 9,12-Octadecadienoic acid, ethyl ester, Ethyl Stearate, Benzyl Laurate, Cinnamyl Phenyl Acetate, Eugenyl Phenyl Acetate, Cinnamyl Cinnamate, Methyl Abietate (Source Pine Resin), Geranyl Palmitate, 2-Methoxy Methyl Benzoate, Allyl Octanoate, Carvyl Propionate, Cyclon Acetate (Manheimer), Dimetol Acetate, Ethyl-3-Hydroxy Hexanoate, Methyl Decadienoate, Propyl Acetate, and t-Butyl Acetate.

Still other exemplary esters include, but are not limited to, 2,4-dimethyl-3-pentyl propionate; 2,4-dimethyl-3-pentyl isobutyrate; 2,4-dimethyl-3-pentyl crotonate; 2,4-dimethyl-3-pentyl butyrate; 2,6-dimethyl-4-heptyl propionate; 2,6-dimethyl-4-heptyl isobutyrate; 2,6-dimethyl-4-heptyl crotonate; 2,6-dimethyl-4-heptyl butyrate; 3,3,5-trimethylcyclohexyl acetate; 3,3,5-trimethylcyclohexyl propionate; 3,3,5-trimethylcyclohexyl crotonate; 3,3,5-trimethylcyclohexyl butyrate; menthyl propionate; menthyl isobutyrate; menthyl crotonate; menthyl butyrate; isomenthyl acetate; isomenthyl propionate; isomenthyl isobutyrate; isomenthyl crotonate; isomenthyl butyrate; isopulegyl propionate; isopulegyl isobutyrate; isopulegyl crotonate; isopulegyl butyrate; 2,6,6-trimethylcycloheptyl acetate; 2,6,6-trimethylcycloheptyl propionate; 2,6,6-trimethylcycloheptyl isobutyrate; 2,6,6-trimethylcycloheptyl crotonate; 2,6,6-trimethylcycloheptyl butyrate; 2,2,4-trimethyl-1,3-pentanediyl diacetate; 2,2,4-trimethyl-1,3-pentanediyl dipropionate; 2,2,4-trimethyl-1,3-pentanediyl diisobutyrate; 2,2,4-trimethyl-1,3-pentanediyl dicrotonate; 2,2,4-trimethyl-1,3-pentanediyl dibutyrate; 2-methyl-2,4-pentanediyl diacetate; 2-methyl-2,4-pentanediyl dipropionate; 2-methyl-2,4-pentanediyl diisobutyrate; and 2-methyl-2,4-pentanediyl dicrotonate.

A malodor control system may comprise odor-masking compounds. Some perfumes exhibit odor-masking properties. This is distinct from the practice of dosing a perfume to overwhelm other odors. Rather, compounds like cyclopentadecanolide, a macrocyclic artificial musk that is also known as muskalactone, 2-Pentadecalone, and cyclopentadecanolactone, may disguise other odors without providing a dominant or overwhelming odor, thereby creating the impression of an overall neutral or slight scent. Other macrocyclic musks include, but are not limited to, 1,4-Dioxacycloheptadecane-5,17-dione, 1,4-Dioxacyclohexadecane-5,16-dione, Oxacycloheptadecan-2-one, Oxacycloheptadec-10-en-2-one, Cyclopentadecan-1-one, 10-Oxahexadecanolide, 11-oxahexadecanolide, Oxacyclohexadecen-2-one, 3-methyl-cyclopentadecan-1-one, 3-methyl-cyclopentadec-4(5)-en-1-one, 5-cyclohexadecen-1-one, and Oxacyclopentadecan-2-one.

Odor-neutralization compounds and odor-masking compounds may be used in conjunction with perfumes or perfume raw materials that are dosed to overwhelm malodors. Such perfumes or perfume raw materials may be added at levels which are not overwhelming, but which reinforce or complement the neutral scent generated by the odor-masking compound.

A malodor control system may comprise odor-reducing compounds. Such compounds may induce a condition known as anosmia—the absence or loss of the sense of smell. If such a compound is used, the anosmia induced may be partial in scope, time, or both. In some embodiments, it may be undesirable to induce complete anosmia for even the relatively short duration of a diaper change (generally on the order of 1-3 minutes). For example, a product comprising a malodor control system may include an appealing perfume or scent associated with the product, and complete anosmia, even if temporary, would eliminate the benefit of the pleasant scent during use.

Exemplary odor-reducing materials are described, for example, in WIPO International Publication No. 2007/113778 to The Procter & Gamble Company, and U.S. Patent Publication No. 2005/0124512 to Woo et al., and may include menthol, menthyl acetate, 3-buten-2-one, 3-methyl-4-(2,6,6-trimethyl-2-cyclohexen-1-yl)-, 4-(2,6,6-trimethylcyclohen-1-en-1-yl)but-2-en-2-one, 3-buten-2-one,4-(2,6,6-trimethyl-2-cyclohexen-1-yl)-,(E)-, menthyl lactate, isomenthyl acetate, isomenthyl propionate, isomenthyl isobutyrate, isomenthyl butyrate, camphor and p-menthane. The materials also include their isomeric forms, diastereomers, and enantiomers. Odor-reducing materials may be selected which do not have strong inherent odors, or which have pleasant inherent odors alone or in combination with other components of the malodor control system or the product in which the malodor control system is ultimately incorporated.

Ionones are a class of compounds which are useful for reducing sensitivity to odors, and are particularly useful in the context of disposable absorbent articles and wipes because ionones block the perception of sulfur smells, which may be associated with some bowel movements. As with other compounds, ionones may be selected based on inherent aromas and other characteristics which vary within the class. Suitable ionones include, for example, alpha-gamma-methyl ionone (CAS Registry No. 127-51-5), beta-gamma-methyl ionone (CAS Registry No. 79-89-0), beta-ionone (CAS Registry No. 14901-07-6), alpha-ionone (CAS Registry No. 127-41-3), methyl-ionone (CAS Registry No. 1335-46-2), and AB-ionone (CAS Registry No. 8013-90-9).

A malodor control system may include an aldehyde, an ester, an ionone, and a macrocyclic musk. In some embodiments, the aldehyde is selected from the group consisting of 2,6 nonadien-1-al, Bourgenal, Citral, Cymal, Decyl Aldehyde, Hexyl cinnamic aldehyde, Laurie aldehyde, Lyral, Melonal, Nonyl aldehyde, Octyl aldehyde, Geranial, Ligustral, Canthoxal, Cyclocitral, 2-methyl pentenal, and mixtures thereof. In some embodiments, the ester is menthyl acetate. The aldehyde(s) may be present at a ratio of between 60:1 and 1:60 relative to the ionone(s), by weight. The ester may be present at a ratio between 100:1 and 1:100 relative to the aldehyde(s), ionone(s), and macrocyclic musk(s) combined. The macrocyclic musk may be present at a ratio between 100:1 and 1:100 relative to the aldehyde(s), ionone(s), and ester(s) combined.

A malodor control system may further comprise a perfume. When used with a perfume, the malodor control system may comprise about 50% or less of the total weight of the malodor control system and perfume combined. At ratios greater than about 50%, such as 60% or 70% or greater, the malodor control system may begin to dominate or overwhelm the perfume, interfering with the overall character of the perfume. In some embodiments, the malodor control system may comprise between 1% and 50%, or between 20% and 50%, or between about 1% and about 20%, of the malodor control system and perfume combined.

When the malodor control system and the perfume are used in a wet wipe, they may comprise a fraction of the lotion or cleansing composition of the wipe. For example, by weight, the malodor control system and perfume combined may comprise less than 10%, less than 5%, or less than 1% of the lotion composition. In the specific case of a baby wipe, the malodor control system and perfume combined may comprise less than 0.1% of the lotion composition, by weight. If no perfume is used, the malodor control system may comprise less than 10%, less than 5%, less than 1% or even less than 0.1% of the lotion composition, by weight.

Some compounds may exhibit activity in more than one of the groups of odor-neutralizing compounds, odor-masking compounds, odor-reducing compounds, and perfumes. For example, menthyl acetate may be used as both an odor-neutralizing compound and an odor-reducing compound.

The selection of compounds included in a baby or cleaning wipe may be guided by a number of commercial and regulatory constraints, which may vary over time and geography, and may include safety, long-term effects of repeated skin exposure to the compound, effects of the compound on other surfaces that might be contacted (i.e., propensity to yellow fabrics, remove paint, or otherwise stain or disfigure hard surfaces), and stability of the compound over time. For example, amylic cinnamic aldehyde and citronellal are aldehydes which serve an odor-neutralization function, but may cause yellowing or browning of some fabrics, whereas citral aldehyde (aliphatic) and lauric aldehyde (aliphatic) are useful odor-neutralizers which may not contribute to yellowing or browning of fabric.

In combination, odor-neutralizing compounds, odor-reducing compounds, and odor-masking compounds may reduce the malodors associated with a diaper change below human-detectable levels. Examples 1-7, as described in detail below, demonstrate that a malodor control system can be adapted to use in a baby wipe with experimentally verifiable efficacy. These results are surprising because it was previously believed that the malodor reduction and control benefits of combined neutralization and odor-control compounds required air dispersal, such as provided by hydrocarbon propellants, mechanical sprays, and liquid mist odor traps, for maximum efficiency. It is therefore surprising that the combination of compounds in these embodiments can reduce malodor below a human-detectable level without airborne dispersal.

These examples therefore describe a combination of components with unexpected benefits. One exemplary advantage of some embodiments is the ability to incorporate a malodor control system into a pre-existing product without substantially changing the perfume or scent profile of the product. That is, where scent is a desirable characteristic of the product itself, a malodor-control function can be conferred using some embodiments without negating or changing the pre-existing product scent profile. Another exemplary advantage of some embodiments is the ability to control the malodors associated with changing a used disposable absorbent article without complicating the procedure through the use of additional products.

### Perfumes

An odor control system may include one or more perfumes. Exemplary scents include, but are not limited to, the seven basic fragrance types or families: Fougere, Green, Chypre, Citrus, Oriental, Fruity, and Floral. Where a wipe employing a malodor control system is designed to be used with coordinated products, the perfume of the wipe may be selected to match, complement, or coordinate with the other product or products. For example, a baby wipe may include the same perfume as a baby diaper, or may include a perfume which has a pleasant smell in combination with a baby diaper, or may include a perfume which has a scent distinct from and complimentary to a perfume in a baby diaper.

### Products Comprising a Malodor Control System

Although the malodor control system is described largely with regard to embodiments involving a wipe or a baby wipe product, it should be appreciated that a malodor control system can be adapted to many other products without undue experimentation. For example, a malodor control system may be contained in a storage unit that can release or aerosolize a small quantity of the malodor control compounds at set time intervals, or upon specified events, such as the opening of a container. Just as a malodor control system can be incorporated into the lotion or cleaning composition of a wipe, a malodor control system may be formulated as a component of a gel or polymer which functions when a storage container comprising the gel or polymer is opened or accessed without being released or aerosolized, that is, without being aerosolized beyond the normal volatility of the components of the malodor control system, such as by spraying, misting, heating or the like.

The malodor control system may be “released” from a container passively over time. For example, the malodor control system may be incorporated into a gel or polymer base which is exposed to ambient air, whereby the components of the malodor control system are volatilized and “released” according to the physical properties (such as boiling point) of the malodor control system. In such an embodiment, the malodor control system would permeate the surrounding air without opening or actuating the container comprising the malodor control system.

Further, the malodor control system need not be used with a nonwoven substrate. For example, the malodor control system may be used with a woven substrate. For example, the malodor control system may be useful in cloth diapers, cleaning cloths, or clothing. As a further example, the malodor control system may be embedded in, disposed on, or otherwise associated with laminate material, a polymer film, polymer particulates, gels, creams, lotions, tapes, thermoplastic compositions, or other substrates.

### Wipes Comprising a Malodor Control System

A wipe may be a particularly effective means of neutralizing odor associated with a surface. Take as an example a baby wipe, used to clean an infant's skin while changing a soiled diaper. The wipe has direct contact with the infant, the caregiver, and at least one source of the malodor, such as urine or fecal residues on the skin. As a result, an effective malodor control system associated with the baby wipe reduces any residual odor from trace residue on the infant's skin, reduces the odor to which the caregiver is exposed while changing the diaper, and reduces the odor from any residues transferred to the wipe when it is used.

A plurality of wipe substrates are known in the art, and disclosed, for example, in U.S. Pat. No. 6,673,358 to Cole et al. and U.S. Patent Publication No. 2007/0286894 to Marsh et al. Disposable wipes are often constructed of nonwoven materials. “Nonwoven” refers herein to a fibrous structure made from an assembly of continuous fibers, coextruded fibers, non-continuous fibers and combinations thereof, without weaving or knitting, by processes such as spunbonding, carding, meltblowing, airlaying, wetlaying, coforming, or other such processes known in the art for such purposes. The process for incorporating a fiber into a substrate may be selected based upon the sorts of component materials used and the desired properties of the substrate web. The nonwoven material may comprise one or more layers of fibrous assemblies, wherein each layer may include continuous fibers, coextruded fibers, non-continuous fibers and combinations thereof.

A suitable wipe may be constructed of any material or blends of material which produce suitable flexibility, durability, and, if desired, liquid absorbency. Suitable fibers may be natural, cellulosic, wholly synthetic, or some combination of fibers. Natural or synthetic fibers may be treated or otherwise modified mechanically or chemically to provide desired characteristics or may be in a form that is generally similar to the form in which they can be found in nature.

In certain embodiments, particular combinations of fibers may be selected to provide desired characteristics. For example, fibers of certain lengths, widths, coarseness, shape or other characteristics may be combined in certain layers or separate from each other. In some embodiments, suitable materials include viscose, polypropylene, polypropylene-viscose blends, cotton, LYOCELL® cellulose fibers, pulp, or a mixture of these materials.

The substrate materials may also be treated to improve the softness and texture thereof. The substrate may be subjected to various treatments, such as, but not limited to, physical treatment, such as hydro-molding, hydro-embossing, ring rolling, as described in U.S. Pat. No. 5,143,679 issued to Weber et al. on Sep. 1, 1992; structural elongation, as described in U.S. Pat. No. 5,518,801 issued to Chappell et al. on May 21, 1996; consolidation, as described in U.S. Pat. Nos. 5,914,084 issued to Benson et al. on Jun. 22, 1999; 6,114,263 issued to Benson et al. on Sep. 5, 2000; 6,129,801 issued to Benson et al. on Oct. 10, 2000 and 6,383,431 issued to Dobrin et al. on May 7, 2002; stretch aperturing, as described in U.S. Pat. Nos. 5,628,097 issued to Benson et al. on May 13, 1997; 5,658,639 issued to Curro et al. on Aug. 19, 1997 and 5,916,661 issued to Benson et al. on Jun. 29, 1999; differential elongation, as described in US Publication No. 2003/0028165A1 published on Feb. 6, 2003 by Curro et al.; and other solid state formation technologies as described in U.S. Publication No. 2004/0131820A1 published on Jul. 8, 2004 by Turner et al. and U.S. Publication No. 2004/0265534A1 published on Dec. 30, 2004 by Curro et al., zone activation, and the like; chemical treatment, such as, but not limited to, rendering part or all of the substrate hydrophobic, and/or hydrophilic, and the like; thermal treatment, such as, but not limited to, thermal-embossing, softening of fibers by heating, thermal bonding and the like; and combinations thereof. Without being bound by theory, it is believed that a textured substrate may further enable the ease of removal of the bodily exudates by improving the ability to grip or otherwise lift the exudates from the skin during cleansing.

The substrate may have a basis weight between about 15, 30, 40 or 45 grams/m² and about 65, 75, 85, 95 or 100 grams/m². One exemplary substrate may be a carded nonwoven comprising a 40/60 blend of viscose fibers and polypropylene fibers having a basis weight of 58 grams/m² as available from Suominen of Tampere, Finland as FIBRELLA™ 3160. Another exemplary material may be FIBRELLA™ 3100 which is a 62 grams/m² nonwoven web comprising 50% w/w 1.5 denier polypropylene fibers and 50% w/w 1.5 denier viscose fibers. Another suitable material for use as a substrate may be SAWATEX™ 2642 as available from Sandler AG of Schwarzenbach/Salle, Germany Yet another suitable material for use as a substrate may have a basis weight of from about 40 grams/m² (gsm) to about 200 gsm and have a 20/80 blend of viscose fibers and polypropylene fibers. The substrate may also be a 60/40 blend of pulp and viscose fibers.

In another embodiment, the substrate may be biodegradable. For example the substrate could be made from a biodegradable material such as a polyesteramide, or a high wet strength cellulose. The substrate may also be dispersible, that is, the substrate or designated portions of the product may sufficiently dissolve or disintegrate in water such that the substrate may be discarded in sewer or septic systems without presenting any problems for typical household or municipal sanitization systems. The materials and methods for making such a dispersible substrate are described, for example, in WO 2007/125443 to Kimberly-Clark Worldwide, Inc.; in U.S. Pat. No. 4,755,421 to Manning et al.; in U.S. Pat. No. 7,285,504 to Jones et al.; in U.S. Pat. No. 7,157,389 to Branham et al.; and in U.S. Pat. No. 7,101,612 to Lang et al.

Other suitable substrates include coform substrates, as described in U.S. Pat. No. 4,100,324 to Anderson et al., and substrates formed by hydrodynamic needling, as described in U.S. Pat. No. 6,842,953 to Orlandi.

### Lotion

Wipes may be impregnated with a liquid or semi-liquid lotion composition intended to enhance cleaning and, for body-contact wipes particularly, to provide a smooth feeling. “Lotion,” as used herein, refers to a composition comprising a carrier such as water or alcohol, and may also be referred to as smoothening lotion, smoothening composition, oil-in-water emulsion composition, emulsion composition, emulsion, or cleaning or cleansing lotion or composition.

Non-limiting examples of suitable lotions are described in U.S. Patent Publication No. 2005/0008681 to Deckner et al.; U.S. Patent Publication No. 2006/0058210 to Marsh et al.; and U.S. Pat. No. 6,673,358 to Cole et al. Additional optional ingredients may be added to the lotion as desired. Some exemplary lotion ingredients are described herein. However, there are a wide variety of known additives useful for a lotion, and most of them are compatible or can be rendered compatible with a malodor control system of the present disclosure through routine experimentation and optimization. The lotion may include a perfume composition, including or in addition to a malodor control system of the present disclosure.

The lotion may be applied to the substrate by dipping, spraying, wicking, submersion, or any other means known in the art for wetting or coating a fibrous substrate. Generally the composition is of sufficiently low viscosity to impregnate the entire structure of the wipe. In some other instances, the composition can be primarily present at the wipe surface and to a lesser extent in the inner structure of the wipe.

The substrate may be fully or partially saturated with the lotion. If partially saturated, the substrate and/or the lotion may be designed such that the lotion resides primarily in defined areas of the wipe. In various embodiments, the substrate may be loaded with between about 1% and about 500% lotion, as weight of lotion to weight of substrate.

In some embodiments, the substrate is substantially unsaturated. For example, the substrate may feel dry to the touch. A malodor control system may be incorporated into such a “dry wipe” using a number of methodologies. For example, a substrate could have a malodor control system applied in liquid form, and then the partially or fully saturated substrate could be dried. In such a process, the volatilization of the malodor control system may be controlled so that the components are available to neutralize odors after processing, packaging, and storage, including storage of any unused product after the package is initially opened by a consumer. This may involve packaging or sealing the product to maintain a headspace and/or inhibit volatilization. In another process, the components of the malodor control system are encapsulated and applied to the substrate. Microencapsulation technologies suitable for use in a skin-contact wipe are described, for example, in U.S. Patent Publication No. 2007/0145326 to Joseph et al.; in U.S. Patent Publication No. 2007/0145617 to Finney et al.; in U.S. Patent Publication No. 2007/0145618 to Finney et al.; in U.S. Patent Publication No. 2007/0145619 to Drath et al.; in U.S. Patent Publication No. 2007/0148446 to Brown et al.; in U.S. Patent Publication No. 2007/0148459 to Joseph et al.; in U.S. Patent Publication No. 2007/0149435 to Koenig et al.; and in U.S. Pat. No. 7,316,994 to Jordan et al.

While encapsulation is particularly useful in constructing a dry wipe, encapsulation may also be useful in a wet wipe. For example, encapsulation may be helpful in maintaining the malodor control components over time; in isolating the malodor control components from other, potentially incompatible compounds in the wipe, in the lotion, or in the environment of the wipe; or in extending the time over which the malodor control components are available to control malodor.

The encapsulated components may be disposed on the surface of the wipe, or may be embedded in the wipe. All of the components of the malodor control system may be encapsulated together, for example, the components may be combined prior to encapsulation, or only select components of the malodor control system may be encapsulated together. In some embodiments, some components are encapsulated and others are not encapsulated. For example, in embodiments where one or more of the selected components of the malodor control system has a relatively high volatility under anticipated use conditions and may dissociate from the wipe prior to use, only the relatively high volatility component or components may be encapsulated. For example, the aldehyde or the ester may be encapsulated. Alternately, the ionone or the macrocyclic musk may be encapsulated. Further, some components may be disposed on the surface of the wipe and others may be embedded in the wipe.

A wipe which is dry when the malodor control components are added to the wipe may also be wetted at a later time, such as at a later stage during manufacturing, after shipping the dry product, or at the point of use.

### Optional Lotion Ingredients

Additional ingredients may be added to the lotion as desired. The lotion may comprise any of the following ingredients: emollients, humectants, surfactants, rheology modifiers, botanicals, skin health agents, anti-stick agents, preservatives, a combination of preservative compounds acting together as a preservative system, or other ingredients. It is to be noted that some ingredient compounds can serve multiple functions and that all compounds are not necessarily present in the composition. The composition may be an aqueous-based solution or an emulsion. The pH of the composition may be from about pH 3, 4, or 5 to about pH 7, 7.5, 8, or 10.

### Emollient

Emollients may (1) improve the glide of the substrate on the skin, by enhancing the lubrication and thus decreasing the abrasion of the skin, (2) hydrate the residues (for example, fecal residues or dried urine residues or menses), thus enhancing their removal from the skin, (3) hydrate the skin, thus reducing its dryness and irritation while improving its flexibility under the wiping movement, and (4) protect the skin from later irritation (for example, caused by the friction of an absorbent article) as the emollient is deposited onto the skin and remains at its surface as a thin protective layer.

Emollients may include silicone oils, functionalized silicone oils, vegetable oils, triglycerides, hydrocarbon oils, fatty alcohols, fatty alcohol ethers, fatty acids, esters of monobasic and/or dibasic and/or tribasic and/or polybasic carboxylic acids with mono and polyhydric alcohols, polyoxyethylenes, polyoxypropylenes, mixtures of polyoxyethylene and polyoxypropylene ethers of fatty alcohols, and mixtures thereof. The emollients may be either saturated or unsaturated, have an aliphatic character and be straight or branched chained or contain alicyclic or aromatic rings. Surprisingly, at least some embodiments of the malodor control system are compatible with fatty acid emollients, even though the malodor control system may include compounds which chemically eliminate the short-chain fatty acids associated with bowel movement malodor. Without wishing to be bound by theory, it is believed that most emollients have longer chain lengths than the malodorous fatty acids, and are therefore not as susceptible to chemical degradation by aldehydes and esters as the malodorous compounds.

A mixture of emollients may be used, for example, caprylic capric triglycerides in combination with Bis-PEG/PPG-16/16 PEG/PPG-16/16 dimethicone known as ABIL CARE™ 85 (available from Degussa Care Specialties of Hopewell, Va.).

Emollients, when present, may be used in a lotion at an emollient weight/lotion weight % from about 0.5%, 1% or 4% to about 0.001%, 0.01%, or 0.02% relative to the lotion composition. All further percentages given are component weight/weight of the composition, unless otherwise noted. Low levels of emollients may reduce the tendency of the emollients to form a greasy or oily layer on the skin.

### Surfactant

The surfactant can be an individual surfactant or a mixture of surfactants. The surfactant may be a polymeric surfactant or a non-polymeric one. The surfactant, when present, may be employed in an amount effective to emulsify the emollient and any other non-water-soluble oils that may be present in the composition, such as an amount ranging from about 0.5%, 1%, or 4% to about 0.001%, 0.01% or 0.02%.

The composition may include one or more surfactants. The surfactant or combinations of surfactants may be mild, which means that the surfactants provide sufficient cleansing or detersive benefits but do not overly dry or otherwise harm or damage the skin.

A wide variety of surfactants are useful herein and include those selected from the group consisting of anionic surfactants, nonionic surfactants, cationic surfactants, amphoteric surfactants, zwitterionic surfactants, silicone surfactants, and mixtures thereof.

Non-limiting examples of anionic surfactants include those selected from the group consisting of sarcosinates, sulfates, sulfonates, isethionates, taurates, phosphates, lactylates, glutamates, and mixtures thereof. Other anionic materials useful herein are soaps (i.e., alkali metal or amine salts, e.g., sodium, potassium or triethanolamine salts) of fatty acids, typically having from about 8 to about 24 carbon atoms.

Nonionic surfactants useful herein include, but are not limited to, those selected from the group consisting of alkyl glucosides, alkyl polyglucosides, polyhydroxy fatty acid amides, alkoxylated fatty acid esters, alkoxylated fatty alcohol ethers, sucrose esters, ethoxylated and propoxylated sucrose esters, ethoxylated and propoxylated mono and diglycerides, amine oxides, block copolymers comprising ethylene oxide/propylene oxide, and mixtures thereof.

Amphoteric surfactants suitable for use in the present compositions are well known in the art and include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group such as carboxy, sulfonate, sulfate, phosphate, or phosphonate. Useful amphoteric surfactants include, but are not limited to, the group consisting of cocoamphoacetate, cocoamphodiacetate, lauroamphoacetate, lauroamphodiacetate, and mixtures thereof.

Zwitterionic surfactants suitable for use herein include those surfactants broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. Useful zwitterionic detersive surfactants are the betaines, amphoacetates and sulfobetaines, e.g., cocoamidopropylbetaine, sodium laurylamphoacetate and cocoamidopropylhydroxysultaine.

### Rheology Modifier

Rheology modifiers are compounds that modify the flow properties of the lotion composition. These materials may also provide “structure” to the compositions to prevent settling out (separation) of insoluble and partially soluble components.

The rheology modifier, in addition to stabilizing the suspension of insoluble and partially soluble components, may also (1) help to stabilize the lotion composition on a substrate, (2) enhance the transfer of the lotion composition to the skin, and (3) enhance the uniformity of the layer of the lotion composition on the skin.

Rheology modifiers may also affect the rheological profile of the lotion composition such that the viscosity of the lotion composition may change as a function of the shear that is applied to the lotion composition. The application of the lotion composition to a surface (e.g. the skin) typically includes a “wiping” or “rubbing” movement. This movement may increase the shear and pressure experienced by the lotion composition. In the case of a shear-thinning rheological profile (i.e. an increase in shear reduces the viscosity of the lotion composition), the viscosity of the lotion may decrease with the increased shear of “wiping” or “rubbing” thereby enabling a better transfer to the skin as well as a better lubrication effect. Additionally, the rheology modifier may help to preserve a homogeneous distribution of the lotion within a stack of the substrates.

Examples of rheology modifiers include, but are not limited to, Ultrez™-10, a carbomer, and Pemulen™ TR-2, an acrylate crosspolymer, both of which are available from Noveon, Cleveland Ohio, and Keltrol™, a Xanthan gum, available from CP Kelco, San Diego Calif., and combinations thereof. Rheology modifiers, when present, may be present from about 0.01%, 0.015%, or 0.02% to about 1%, 2% or 3%.

### Preservative

Preservatives may be used to control microbiological growth in a liquid composition. Where a malodor control system is used with a wipe, the lotion may comprise a preservative or a combination of preservatives acting together as a preservative system. Preservatives and preservative systems are used interchangeably in the present document to indicate one unique preservative compound or a combination of preservative compounds. A preservative may be understood to be a compound or a combination of compounds reducing the growth of microorganisms, thus enabling a longer shelf life for a package of substrates (opened or not opened) as well as creating an environment with reduced growth of microorganisms when transferred to the skin during the wiping process.

The spectrum of activity of the preservative may include bacteria, molds and yeast. Each of such microorganisms may be killed by the preservative. Another mode of action to be contemplated may be the reduction of the growth rate of the microorganisms without active killing. Materials useful as preservatives include, but are not limited to: methylol compounds, iodopropynyl compounds, simple aromatic alcohols, paraben compounds, chelators such as ethylenediamine tetraacetic acid, and combinations thereof.

Methylol compounds can be used at concentrations between about 0.025% and about 0.50%. In another embodiment, the concentration may be about 0.075%. The iodopropynyl compound may provide antifungal activity. Iodopropynyl compounds can be used effectively at a concentration between about 0.001% and about 0.05%. A preservative system of this type may comprise a blend of a methylol compound at a concentration of about 0.075% and an iodopropynyl compound at a concentration of about 0.009%.

In another embodiment, the preservative system may comprise simple aromatic alcohols (e.g. benzyl alcohol). Materials of this type may have effective antibacterial activity. Benzyl alcohol is available from Symrise, Inc. of Teterboro, N.J.

In another embodiment, the preservative may comprise at least one paraben antimicrobial. The preservative may be a paraben antimicrobial selected from the group consisting of methylparaben, ethylparaben, propylparaben, butylparaben, isobutylparaben or combinations thereof. The total concentration of paraben antimicrobial may be lower than about 0.3%, 0.5%, or 1%. The minimum amount of paraben antimicrobial may be any amount sufficient to obtain the desired preservation of the composition, such as more than about 0.001%.

In another embodiment, acidic compounds used in sufficient amount to reduce the pH of the lotion composition (e.g. pH of less than about 5) may be useful as the preservative, or as a potentiator for other preservative ingredients.

### Adjunct Ingredients

The lotion composition may optionally include other adjunct ingredients. Possible adjunct ingredients may be selected from a wide range of additional ingredients such as, but not limited to, humectants, botanicals, texturizers, colorants, soothing agents and medically active ingredients, such as healing actives and skin protectants. Materials which reduce the adherence of bodily exudates to the skin may also be desirable. Such materials are described, for example, in U.S. Patent Publication Nos. 2007/0286893 and 2007/0286894, both to Marsh et al.

### Malodor Evaluation Test

For each malodor control compound tested, the malodor formulation is compounded by mixing together 40 mL distilled water, 5 μL synthetic urine malodor, and 15 μL, synthetic fecal malodor at 68-75° F. and 40-50% RH (simulating typical air conditioned room temperature and humidity). The synthetic (human) urine malodor and synthetic (human) fecal malodor are available from Givaudan Fragrances Corporation of Teaneck, N.J. For each test, 0.50 mL of malodor formulation is placed in a 600 mL beaker, Kimax No. 14000, available from VWR as catalog #89003-794, just before use. A malodor control system described herein is blended into a lotion and loaded onto a nonwoven substrate, specifically a blend of spunlaced polypropylene and cotton (60:40 ratio) having a basis weight of 68 gsm. The substrate is loaded with 340% lotion, as weight of lotion/weight of substrate, as modified by each example. The generally square substrate has dimensions of approximately 180 mm×180 mm, resulting in a lotion load of ˜7.5 g of lotion per wipe. Testing is performed in an ELECTROTECH environmental chamber of ˜13 ft³ held at 68-75° F. and 40-50% RH (simulating typical air conditioned room temperature and humidity).

The beaker containing the malodor formulation and/or test formula is placed in the chamber through the front door. Malodor in the chamber is evaluated by sniffing through the front door/sniff port, which is closed between evaluations. Between runs, the chamber is cleaned with hot water mixed with 1% unfragranced dishwashing liquid and rinsed with 91% rubbing alcohol. The chamber is then air dried with the door open until no detectable odor is present, approximately 30-60 minutes, before using the chamber for a new test.

For each test, the malodor formulation is added to a beaker, and the beaker is swirled so the solution covers the base of the beaker. The beaker is placed in an environmental chamber and a baseline intensity grade is assigned by trained perfumers after 1-2 minutes based on the following scale:

MALODOR EVALUATION SCALE

| | |
|---|---|
| 0 | NO MALODOR PRESENT |
| 10 | I THINK THERE IS MALODOR PRESENT (UNSURE) |
| 25 | SLIGHT MALODOR PRESENT |
| 50 | MODERATE MALODOR PRESENT |
| 75 | STRONG MALODOR PRESENT |
| 100 | EXTREMELY STRONG MALODOR PRESENT |

The bottom of the beaker is then cleaned using one test wipe, wiping the wipe against the bottom of the beaker with a circular motion repeated for three rotations using a pressure similar to that used while cleaning a baby's bottom with a wipe. The “cleaned” beaker is then placed in a second, fresh environmental chamber (the “dirty” wipe is left in the first environmental chamber). Both chambers are then evaluated and assigned a malodor grade at 1 minute, 5 minute, and 15 minute intervals. All steps are then repeated for each additional test sample. At least 2 trained perfumers participate in each test to make the test as sensitive and repeatable as possible.

### EXAMPLES

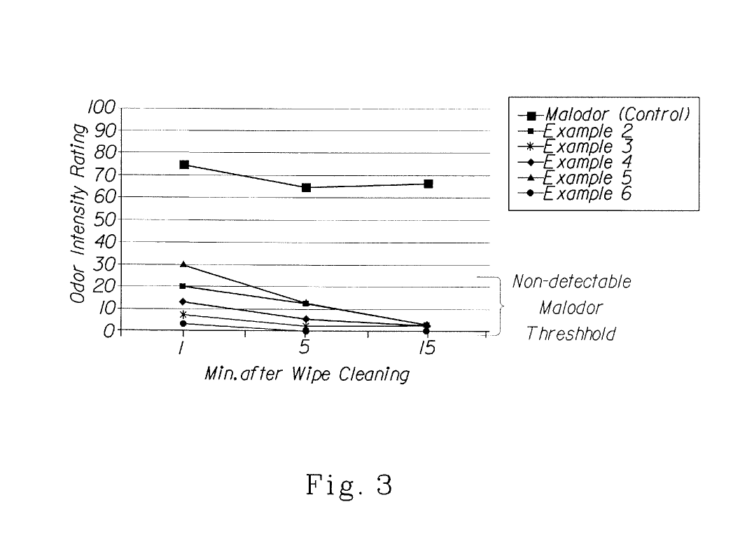

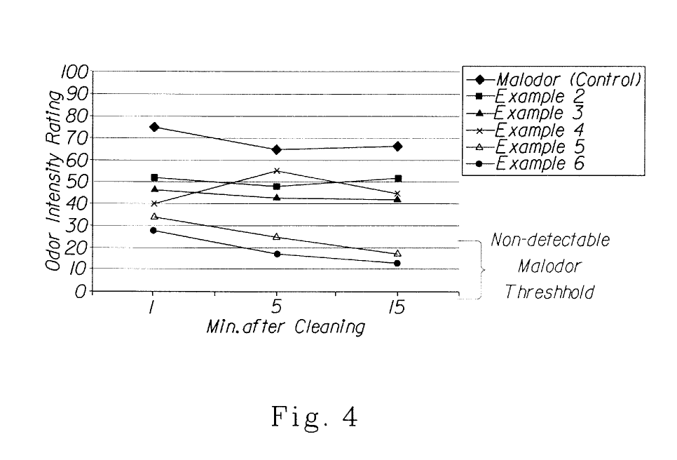

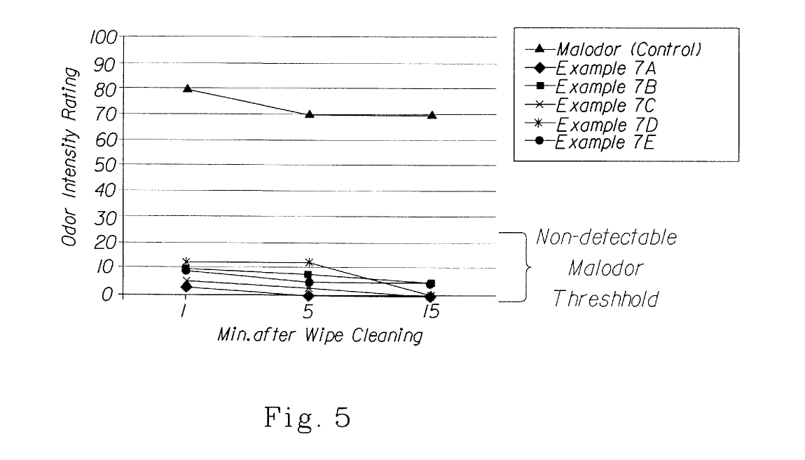

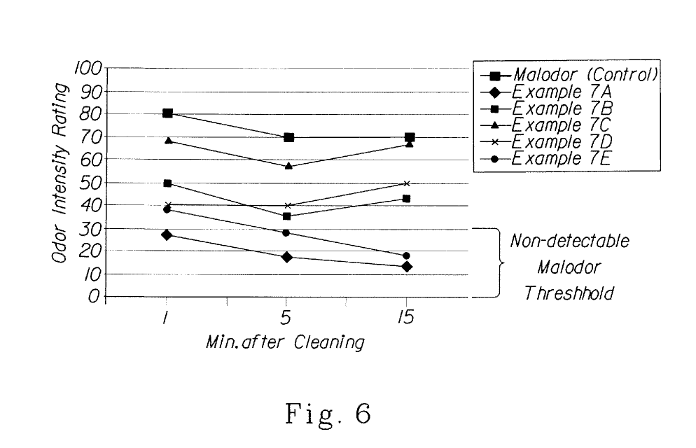

Examples are constructed and tested according to the malodor evaluation test described above. The results of testing Examples 1-6 and 7A-E are shown in FIGS. 3-6, from which it can be seen that a malodor control system works significantly better than any of its constituent parts, including sub-combinations of constituents. Surprisingly, the combination of constituents described herein not only reduces bowel movement and urine malodor better than its constituent parts, it also reduces bowel movement and urine malodor below a human-detectable threshold level.

### Example 1

Malodor formulation—the malodor formulation alone (untreated) is consistently assigned an average odor intensity rating greater than 60, with an initial rating greater than 70. There is no significant decrease in the odor intensity rating of the untreated malodor formulation between the 5 and 15 minute intervals.

### Example 2

A baby wipe with a lotion formula containing 0.005% aldehydes is used to clean the bottom of the beaker. The odor intensity rating of the dirty wipe is reduced to approximately 50 throughout the 15 minute measurement period. The odor intensity rating of the cleaned beaker (simulating a baby's wiped skin) is reduced below the detectable threshold (25) and remains below detectable levels for the 15 minute testing period.

### Example 3

A baby wipe with a lotion formula containing 0.01% EXALTOLIDE is used to clean the bottom of the beaker. The odor intensity rating of the dirty wipe is reduced to approximately 45 throughout the 15 minute measurement period. The odor intensity rating of the cleaned beaker (simulating a baby's skin) is reduced below the detectable threshold (25) and remains below detectable levels for the 15 minute testing period.

### Example 4

A baby wipe with a lotion formula containing 0.005% Odor Neutralizer D61012d (available from Symrise Inc. of Teterboro, N.J.) is used to clean the bottom of the beaker. The odor intensity rating of the dirty wipe drops to 40 after 1 minute, returns to approximately 55 after 5 minutes, and drops again to approximately 45 after 15 minutes. The odor intensity rating of the cleaned beaker (simulating a baby's skin) is reduced below the detectable threshold (25) and remains below detectable levels for the 15 minute testing period.

### Example 5

A baby wipe with a lotion formula containing 0.03% ionone is used to clean the bottom of the beaker. The odor intensity rating of the dirty wipe drops to approximately 35 after 1 minute, and continues to drop throughout the test period, ending just under 20 after 15 minutes. The odor intensity rating of the cleaned beaker (simulating the baby's skin) drops to 30 after 1 minute, and by 5 minutes drops below the detectable threshold. The odor intensity rating of the cleaned beaker remains below the detectable threshold at 15 minutes.

### Example 6

A baby wipe with a lotion formula containing 0.05% of a malodor control system (60% ionone, 10% aldehyde, 20% EXALTOLIDE and 10% Odor Neutralizer D61012d) is used to clean the bottom of the beaker. The odor intensity rating of the dirty wipe is reduced to approximately 25 within 1 minute of cleaning, and continues to decline to just over 10 after 15 minutes. The odor intensity rating of the cleaned beaker (simulating a baby's skin) is reduced below the detectable threshold (25) and remains below detectable levels for the 15 minute testing period.

### Example 7

Baby wipe with lotion formulas containing (A) 0.05% malodor control system (60% ionone, 10% aldehyde, 20% EXALTOLIDE and 10% Odor Neutralizer D61012d); (B) 0.05% parent perfume; (C) 0.05% of a 1:3 mixture of ionones and aldehydes; (D) 0.05% Odor Neutralizer D61012d; and (E) 0.05% of an 80:20 mixture of malodor control system and a base perfume typical of commercially available wipes were produced. When the cleaned beakers are evaluated, all test formulations are below the detectable threshold; the control odor intensity rating remains at or above 70 for the 15 minute testing period. The cleaned beakers, therefore, may serve as a sort of control to verify that the beakers were effectively wiped with each test wipe. When the dirty wipes are evaluated (in the original chambers), the wipe having a malodor control system has an odor intensity rating just above the detectable threshold after 1 minute, and below the detectable threshold for the remainder of the test period. The wipe having a malodor control system-perfume mixture drops below the detectable threshold at the 15 minute test point. In contrast, the wipes containing all other formulas have detectable malodor throughout the 15 minute test period.

Tables 1 and 2 summarize Examples 2-7. Table 1 also includes Examples 8-9, which are conceptual examples of other malodor control systems as disclosed herein.

TABLE 1

| Component | Ex. 2 | Ex. 3 | Ex. 4 | Ex. 5 | Ex. 6 | Ex. 8 | Ex. 9 |
|---|---|---|---|---|---|---|---|
| 2,6 Nonadien-1-al | X | | | | X | X | |
| 2-methyl pentenal | | | | | | X | X |
| Bourgenal | X | | | | X | X | X |
| Canthoxal | | | | | | X | X |
| Citral | X | | | | X | X | |
| Cyclocitral | | | | | | X | X |
| Cymal | X | | | | X | X | X |
| Ligustral | | | | | | X | X |
| Decyl Aldehyde | X | | | | X | X | |
| Geranial | | | | | | X | X |
| Hexyl Cinnamic | X | | | | X | X | |
| Aldehyde | | | | | | | |
| Lauric Aldehyde | X | | | | X | X | |
| Lyral | X | | | | X | X | X |
| Melonal | X | | | | X | X | X |
| Nonyl Aldehyde | X | | | | X | X | |
| Octyl Aldehyde | X | | | | X | X | |
| Lilial | X | | | | X | X | X |
| EXALTOLIDE | | X | | | X | | X |
| macrocyclic musk | | | | | | | |
| Odor Neutralizer | | | X | | X | X | |
| D61012d | | | | | | | |
| Alpha-Ionone | | | | X | X | X | X |
| Gamma methyl | | | | | | | |
| Beta-Ionone | | | | X | X | X | X |
| Gamma methyl | | | | | | | |
| Ionone Alpha | | | | X | X | X | X |
| Ionone Beta | | | | X | X | X | X |
| Methyl Ionone | | | | X | X | X | X |
| Ionone AB | | | | X | X | X | X |
| Lotion Composition | X | X | X | X | X | X | X |
| (excluding other | | | | | | | |
| perfume raw | | | | | | | |
| materials or | | | | | | | |
| perfumes) | | | | | | | |

TABLE 2

| Component | Ex. 7A | Ex. 7B | Ex. 7C | Ex. 7D | Ex. 7E |
|---|---|---|---|---|---|
| 2,6 Nonadien-1-al | X | | X | | X |
| Bourgenal | | | | | |
| Citral | | | | | |
| Cymal | | | | | |
| Decyl Aldehyde | | | | | |
| Hexyl Cinnamic | | | | | |
| Aldehyde | | | | | |
| Lauric Aldehyde | | | | | |
| Lyral | | | | | |
| Melonal | | | | | |
| Nonyl Aldehyde | | | | | |
| Octyl Aldehyde | | | | | |
| Lilial | | | | | |
| EXALTOLIDE | X | | | | X |
| macrocyclic musk | | | | | |
| Odor Neutralizer | X | | | X | X |
| D61012d | | | | | |
| Alpha-Ionone | X | | X | | X |
| Gamma methyl | | | | | |
| Beta-Ionone | X | | X | | X |
| Gamma methyl | | | | | |
| Ionone Alpha | X | | X | | X |
| Ionone Beta | X | | X | | X |
| Methyl Ionone | X | | X | | X |
| Ionone AB | X | | X | | X |
| Parent Perfume | | X | | | X |
| Lotion Composition | X | X | X | X | X |
| (excluding other | | | | | |
| perfume raw | | | | | |
| materials or perfumes) | | | | | |

EXAMPLES 10-60, described in Tables 3-10 below, are conceptual examples showing how a malodor control system might be incorporated into various exemplary lotion compositions suitable for use with a baby wipe.

TABLE 3

| | Weight Percent | | | | | | |
|---|---|---|---|---|---|---|---|
| Components | Ex. 10 | Ex. 11 | Ex. 12 | Ex. 13 | Ex. 14 | Ex. 15 | Ex. 16 |
| Water | Q.S. | Q.S. | Q.S. | Q.S. | Q.S. | Q.S. | Q.S. |
| Disodium EDTA | 0.100 | 0.100 | 0.100 | 0.100 | 0.100 | 0.100 | 0.100 |
| Iodopropynylbutylcarbamate | 0.009 | 0.009 | 0.009 | 0.009 | 0.009 | 0.009 | 0.009 |
| Benzyl Alcohol | 0.500 | 0.500 | 0.500 | 0.500 | 0.500 | 0.500 | 0.500 |
| Suttocide ® A 50% Solution | 0.150 | 0.150 | 0.150 | 0.150 | 0.150 | 0.150 | 0.150 |
| Xanthan Gum | 0.200 | 0.200 | 0.200 | 0.200 | 0.200 | 0.200 | 0.200 |
| PEG-40 Hydrogenated | 0.440 | 0.440 | 0.440 | 0.440 | 0.440 | 0.440 | 0.440 |
| Castor Oil | | | | | | | |
| Abil Care 85 | 0.100 | 0.450 | 0.100 | 0.450 | 0.100 | 0.450 | 0.450 |
| Citric Acid | 0.055 | 0.055 | 0.055 | 0.055 | 0.055 | 0.055 | 0.055 |
| Glycerin | 0.000 | 0.000 | 1.000 | 1.000 | 0.000 | 0.000 | 1.000 |
| Sodium PEG-8 Phosphate | 0.000 | 0.000 | 0.000 | 0.000 | 2.000 | 2.000 | 2.000 |
| Malodor Control System | 0.050 | 0.050 | 0.050 | 0.050 | 0.050 | 0.050 | 0.050 |

TABLE 4

| | Weight Percent | | | | | | |
|---|---|---|---|---|---|---|---|
| Components | Ex. 17 | Ex. 18 | Ex. 19 | Ex. 20 | Ex. 21 | Ex. 22 | Ex. 23 |
| Water | Q.S. | Q.S. | Q.S. | Q.S. | Q.S. | Q.S. | Q.S. |
| Disodium EDTA | 0.100 | 0.100 | 0.100 | 0.100 | 0.100 | 0.100 | 0.100 |
| PEG-40 Hydrogenated | 0.400 | 0.400 | 0.400 | 0.400 | 0.400 | 0.400 | 0.400 |
| Castor Oil | | | | | | | |
| Propylene Glycol | 1.500 | 1.500 | 1.500 | 1.500 | 1.500 | 1.500 | 1.500 |
| Phenoxyethanol | 0.800 | 0.800 | 0.800 | 0.800 | 0.800 | 0.800 | 0.800 |
| Methyl Paraben | 0.150 | 0.150 | 0.150 | 0.150 | 0.150 | 0.150 | 0.150 |
| Ethyl Paraben | 0.050 | 0.050 | 0.050 | 0.050 | 0.050 | 0.050 | 0.050 |
| Propyl Paraben | 0.050 | 0.050 | 0.050 | 0.050 | 0.050 | 0.050 | 0.050 |
| Xanthan Gum | 0.200 | 0.200 | 0.200 | 0.200 | 0.200 | 0.200 | 0.200 |
| Abil Care 85 | 0.100 | 0.450 | 0.100 | 0.450 | 0.100 | 0.450 | 0.450 |
| Trilaureth-4 Phosphate | 0.400 | 0.400 | 0.400 | 0.400 | 0.400 | 0.400 | 0.400 |
| Monobasic Sodium Phosphate | 0.180 | 0.180 | 0.180 | 0.180 | 0.180 | 0.180 | 0.180 |
| Glycerin | 0.000 | 0.000 | 1.000 | 1.000 | 0.000 | 0.000 | 1.000 |
| Sodium PEG-8 Phosphate | 0.000 | 0.000 | 0.000 | 0.000 | 2.000 | 2.000 | 2.000 |
| Malodor Control System | 0.050 | 0.050 | 0.050 | 0.050 | 0.050 | 0.050 | 0.050 |

TABLE 5

| | Weight Percent | | | | | | |
|---|---|---|---|---|---|---|---|
| Components | Ex. 24 | Ex. 25 | Ex. 26 | Ex. 27 | Ex. 28 | Ex. 29 | Ex. 30 |
| Water | Q.S. | Q.S. | Q.S. | Q.S. | Q.S. | Q.S. | Q.S. |
| Disodium EDTA | 0.100 | 0.100 | 0.100 | 0.100 | 0.100 | 0.100 | 0.100 |
| Xanthan Gum | 0.200 | 0.200 | 0.200 | 0.200 | 0.200 | 0.200 | 0.200 |
| Abil Care 85 | 0.100 | 0.450 | 0.100 | 0.450 | 0.100 | 0.450 | 0.450 |
| Sodium Benzoate | 0.120 | 0.120 | 0.120 | 0.120 | 0.120 | 0.120 | 0.120 |
| PEG-40 Hydrogenated | 0.440 | 0.440 | 0.440 | 0.440 | 0.440 | 0.440 | 0.440 |
| Castor Oil | | | | | | | |
| Citric Acid | 0.530 | 0.530 | 0.530 | 0.530 | 0.530 | 0.530 | 0.530 |
| Trisodium Citrate | 0.330 | 0.330 | 0.330 | 0.330 | 0.330 | 0.330 | 0.330 |
| Benzyl Alcohol | 0.300 | 0.300 | 0.300 | 0.300 | 0.300 | 0.300 | 0.300 |
| Euxyl ® PE9010^{Δ} | 0.300 | 0.300 | 0.300 | 0.300 | 0.300 | 0.300 | 0.300 |
| Glycerin | 0.000 | 0.000 | 1.000 | 1.000 | 0.000 | 0.000 | 1.000 |
| Sodium PEG-8 Phosphate | 0.000 | 0.000 | 0.000 | 0.000 | 2.000 | 2.000 | 2.000 |
| Malodor Control System | 0.050 | 0.050 | 0.050 | 0.050 | 0.050 | 0.050 | 0.050 |

^{Δ}As available from Schuelke and Mayr GmbH of Norderstedt, Germany.

TABLE 6

| | Weight Percent | | | |
|---|---|---|---|---|
| Components^{†} | Ex. 31 | Ex. 32 | Ex. 33 | Ex. 34 |
| Water | Q.S. | Q.S. | Q.S. | Q.S. |
| Potassium Laureth Mono | 0.600 | 0.000 | 0.000 | 0.000 |
| Alkyl Phosphate^{Δ} | | | | |
| Potassium Coco Mono Alkyl | 0.000 | 0.400 | 0.000 | 0.000 |
| Phosphate^{Δ} | | | | |
| Potassium Ceteth Mono | 0.000 | 0.000 | 0.500 | 0.000 |
| Alkyl Phosphate^{Δ} | | | | |
| Potassium Behenyl Mono | 0.000 | 0.000 | 0.000 | 0.400 |
| Alkyl Phosphate^{Δ} | | | | |
| Glycerin | 0.290 | 0.000 | 0.000 | 0.000 |
| Propylene Glycol | 0.000 | 0.500 | 0.500 | 0.500 |
| Polysorbate-20 | 0.300 | 0.300 | 0.000 | 0.300 |
| Sodium Hydroxymethylglycinate | 0.200 | 0.150 | 0.150 | 0.200 |
| Propyl Paraben | 0.100 | 0.000 | 0.000 | 0.100 |
| Malodor Control System | 0.050 | 0.050 | 0.050 | 0.050 |

^{Δ}Available from Rhodia Novecare of Cranbury, NJ, under the trade designation RHODAFAC
^{†}Solution pH may optionally be adjusted using malic acid

TABLE 7

| | Weight Percent | | | | | |
|---|---|---|---|---|---|---|
| Components | Ex. 35 | Ex. 36 | Ex. 37 | Ex. 38 | Ex. 39 | Ex. 40 |
| Water | Q.S. | Q.S. | Q.S. | Q.S. | Q.S. | Q.S. |
| Disodium EDTA | 0.100 | 0.100 | 0.100 | 0.100 | 0.100 | 0.100 |
| Iodopropynylbutylcarbamate | 0.009 | 0.009 | 0.009 | 0.009 | 0.009 | 0.009 |
| Benzyl Alcohol | 0.500 | 0.500 | 0.500 | 0.500 | 0.500 | 0.500 |
| Suttocide ® A 50% Solution | 0.150 | 0.150 | 0.150 | 0.150 | 0.150 | 0.150 |
| Xanthan Gum | 0.200 | 0.200 | 0.200 | 0.200 | 0.200 | 0.200 |
| PEG-40 Hydrogenated | 0.440 | 0.440 | 0.440 | 0.440 | 0.440 | 0.440 |
| Castor Oil | | | | | | |
| Abil Care 85 | 0.100 | 0.100 | 0.450 | 0.100 | 0.100 | 0.450 |
| Citric Acid | 0.055 | 0.055 | 0.055 | 0.055 | 0.055 | 0.055 |
| Glycerin | 0.000 | 0.000 | 0.000 | 0.000 | 0.000 | 0.000 |
| Sodium PEG-8 Phosphate | 0.000 | 2.000 | 2.000 | 0.000 | 2.000 | 2.000 |
| Malodor Control System | 0.010 | 0.010 | 0.010 | 0.100 | 0.100 | 0.100 |

TABLE 8

| | Weight Percent | | | | | |
|---|---|---|---|---|---|---|
| Components | Ex. 41 | Ex. 42 | Ex. 43 | Ex. 44 | Ex. 45 | Ex. 46 |
| Water | Q.S. | Q.S. | Q.S. | Q.S. | Q.S. | Q.S. |
| Disodium EDTA | 0.100 | 0.100 | 0.100 | 0.100 | 0.100 | 0.100 |
| PEG-40 Hydrogenated | 0.400 | 0.400 | 0.400 | 0.400 | 0.400 | 0.400 |
| Castor Oil | | | | | | |
| Propylene Glycol | 1.500 | 1.500 | 1.500 | 1.500 | 1.500 | 1.500 |
| Phenoxyethanol | 0.800 | 0.800 | 0.800 | 0.800 | 0.800 | 0.800 |
| Methyl Paraben | 0.150 | 0.150 | 0.150 | 0.150 | 0.150 | 0.150 |
| Ethyl Paraben | 0.050 | 0.050 | 0.050 | 0.050 | 0.050 | 0.050 |
| Propyl Paraben | 0.050 | 0.050 | 0.050 | 0.050 | 0.050 | 0.050 |
| Xanthan Gum | 0.200 | 0.200 | 0.200 | 0.200 | 0.200 | 0.200 |
| Abil Care 85 | 0.100 | 0.100 | 0.450 | 0.100 | 0.100 | 0.450 |
| Trilaureth-4 Phosphate | 0.400 | 0.400 | 0.400 | 0.400 | 0.400 | 0.400 |
| Monobasic Sodium Phosphate | 0.180 | 0.180 | 0.180 | 0.180 | 0.180 | 0.180 |
| Glycerin | 0.000 | 0.000 | 0.000 | 0.000 | 0.000 | 0.000 |
| Sodium PEG-8 Phosphate | 0.000 | 2.000 | 2.000 | 0.000 | 2.000 | 2.000 |
| Malodor Control System | 0.010 | 0.010 | 0.010 | 0.100 | 0.100 | 0.100 |

TABLE 9

| | Weight Percent | | | | | |
|---|---|---|---|---|---|---|
| Components | Ex. 47 | Ex. 48 | Ex. 49 | Ex. 50 | Ex. 51 | Ex. 52 |
| Water | Q.S. | Q.S. | Q.S. | Q.S. | Q.S. | Q.S. |
| Disodium EDTA | 0.100 | 0.100 | 0.100 | 0.100 | 0.100 | 0.100 |
| Xanthan Gum | 0.200 | 0.200 | 0.200 | 0.200 | 0.200 | 0.200 |
| Abil Care 85 | 0.100 | 0.100 | 0.450 | 0.100 | 0.100 | 0.450 |
| Sodium Benzoate | 0.120 | 0.120 | 0.120 | 0.120 | 0.120 | 0.120 |
| PEG-40 Hydrogenated | 0.440 | 0.440 | 0.440 | 0.440 | 0.440 | 0.440 |
| Castor Oil | | | | | | |
| Citric Acid | 0.530 | 0.530 | 0.530 | 0.530 | 0.530 | 0.530 |
| Trisodium Citrate | 0.330 | 0.330 | 0.330 | 0.330 | 0.330 | 0.330 |
| Benzyl Alcohol | 0.300 | 0.300 | 0.300 | 0.300 | 0.300 | 0.300 |
| Euxyl ® PE9010^{Δ} | 0.300 | 0.300 | 0.300 | 0.300 | 0.300 | 0.300 |
| Glycerin | 0.000 | 0.000 | 0.000 | 0.000 | 0.000 | 0.000 |
| Sodium PEG-8 Phosphate | 0.000 | 2.000 | 2.000 | 0.000 | 2.000 | 2.000 |
| Malodor Control System | 0.010 | 0.010 | 0.010 | 0.100 | 0.100 | 0.100 |

^{Δ}As available from Schuelke and Mayr GmbH of Norderstedt, Germany.

TABLE 10

| | Weight Percent | | | | | | | |
|---|---|---|---|---|---|---|---|---|
| Components^{†} | Ex. 53 | Ex. 54 | Ex. 55 | Ex. 56 | Ex. 57 | Ex. 58 | Ex. 59 | Ex. 60 |
| Water | Q.S. | Q.S. | Q.S | Q.S. | Q.S. | Q.S. | Q.S. | Q.S. |
| Potassium Laureth Mono | 0.600 | 0.000 | 0.000 | 0.000 | 0.600 | 0.000 | 0.000 | 0.000 |
| Alkyl Phosphate^{Δ} | | | | | | | | |
| Potassium Coco Mono | 0.000 | 0.400 | 0.000 | 0.000 | 0.000 | 0.400 | 0.000 | 0.000 |
| Alkyl Phosphate^{Δ} | | | | | | | | |
| Potassium Ceteth Mono | 0.000 | 0.000 | 0.500 | 0.000 | 0.000 | 0.000 | 0.500 | 0.000 |
| Alkyl Phosphate^{Δ} | | | | | | | | |
| Potassium Behenyl Mono | 0.000 | 0.000 | 0.000 | 0.400 | 0.000 | 0.000 | 0.000 | 0.400 |
| Alkyl Phosphate^{Δ} | | | | | | | | |
| Glycerin | 0.290 | 0.000 | 0.000 | 0.000 | 0.290 | 0.000 | 0.000 | 0.000 |
| Propylene Glycol | 0.000 | 0.500 | 0.500 | 0.500 | 0.000 | 0.500 | 0.500 | 0.500 |
| Polysorbate-20 | 0.300 | 0.300 | 0.000 | 0.300 | 0.300 | 0.300 | 0.000 | 0.300 |
| Sodium | 0.200 | 0.150 | 0.150 | 0.200 | 0.200 | 0.150 | 0.150 | 0.200 |
| Hydroxymethylglycinate | | | | | | | | |
| Propyl Paraben | 0.100 | 0.000 | 0.000 | 0.100 | 0.100 | 0.000 | 0.000 | 0.100 |
| Malodor Control System | 0.010 | 0.010 | 0.010 | 0.010 | 0.100 | 0.100 | 0.100 | 0.100 |

^{Δ}Available from Rhodia Novecare of Cranbury, NJ, under the trade designation RHODAFAC
^{†}Solution pH may optionally be adjusted using malic acid

### Example 61

A malodor control system according to the present disclosure was evaluated for odor control and presentation of a pleasant smell (preservation of perfume perception) over time at body temperatures. A wipe was made according to Example 2, above. A commercially available wipe not having a malodor control system according to the present disclosure was used as a control. One μl of synthetic urine malodor was mixed in 1000 g distilled water. The malodor was then placed in a beaker (150 mL). The wipe to be tested is stretched over the top of the beaker, along with three layers of paper towels and fastened with a rubber band. The beaker is then placed in an environmental chamber fitted over a laboratory hot plate to maintain a temperature of 48±2° C. This equates to approximately the human body temperature of 37° C. (98.6° F.) in the head space where the odor evaluations occur. Evaluations of malodor and pleasant smell intensity were made by expert perfumers initially (0 hours), at 4 hours, and at 8 hours. The wipe having a malodor control system according to the present disclosure consistently had a lower malodor intensity and a higher pleasant smell intensity over the 8 hour period. This test demonstrates that the use of the malodor control system of the present disclosure is compatible with perfumes, and may even enhance the perception of pleasant smells even in the presence of urine malodor.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A malodor control system comprising:
an aldehyde;
an ester;
an ionone; and
a macrocyclic musk.

2. A wipe comprising a nonwoven substrate and the malodor control system of claim 1.

3. The wipe of claim 2, further comprising a lotion composition.

4. The wipe of claim 3, wherein the wipe is at least partially saturated with the lotion composition.

5. The wipe of claim 3, wherein the wipe is fully saturated with the lotion composition.

6. The wipe of claim 2, wherein the wipe is substantially unsaturated.

7. The wipe of claim 2, wherein the ionone and the macrocyclic musk are encapsulated.

8. The wipe of claim 2, wherein the aldehyde or the ester is encapsulated.

9. The wipe of claim 7, wherein the ionone and the macrocyclic musk are at least partially embedded in the nonwoven substrate.

10. The wipe of claim 3, wherein the ionone and the macrocyclic musk are encapsulated.

11. The wipe of claim 2, further comprising a perfume.

12. The wipe of claim 11, wherein the aldehyde, the ester, the ionone, and the macrocyclic musk are incorporated into a perfume blend, and wherein the aldehyde, the ester, the ionone, and the macrocyclic musk combined are less than 20% by weight of the perfume blend.

13. The wipe of claim 2, wherein the wipe can reduce the odor intensity rating of simulated bowel movement and urine malodor below 25 within 5 minutes of exposure.

14. A container comprising the malodor control system of claim 1.

15. The container of claim 14 wherein the malodor control system is in the form of a gel, particles, polymer, laminate, film, or tape.

16. The container of claim 14 wherein the malodor control system is passively released.

17. The container of claim 16 wherein the malodor control system is passively released upon opening the container.

18. The malodor control system of claim 1, comprising one or more aldehydes and one or more ionones, wherein the aldehyde or aldehydes are present at a ratio of between 60:1 and 1:60 relative to the ionone or ionones, by weight.

19. The malodor control system of claim 1, comprising one or more esters, one or more aldehydes, one or more ionones, and one or more macrocyclic musks, wherein the esters are present at a ratio between 100:1 and 1:100 relative to the aldehydes, ionones, and macrocyclic musks combined.

20. The malodor control system of claim 1, comprising one or more macrocyclic musks, one or more aldehydes, one or more ionones, and one or more esters, wherein the macrocyclic musks are present at a ratio between 100:1 and 1:100 relative to the aldehydes, ionones, and esters combined.

21. The wipe of claim 3, wherein the lotion composition comprises an emollient.
